(19) United States
(12) Patent Application Publication
(10) Pub. No.: US 2010/0303933 A1
(43) Pub. Date: Dec. 2, 2010

(54) IMMUNOPOTENTIATING COMPOSITION FROM LABISIA PUMILA EXTRACT

(75) Inventors: Ghulam Nabi Qazi, Jammu Tawi (IN); Sarang Bani, Jammu Tawi (IN); Anpurna Kaull, Jammu Tawi (IN); Anjali Pandey, Jammu Tawi (IN); Kiranjeet Kour, Jammu Tawi (IN); Surrinder Koul, Jammu Tawi (IN); Bishan Dutt Gupta, Jammu Tawi (IN); Pyrelal Sangwan, Jammu Tawi (IN); Ravinder Kumar Raina, Jammu Tawi (IN); Rajendran Manickavasagar, Petaling Jaya (MY)

Correspondence Address: WORKMAN NYDEGGER;1000 Eagle Gate Tower, 60 East South Temple, Salt Lake City, UT, 84111, US

(73) Assignees: HOLISTA BIOTECH SDN. BHD., Petaling Jaya (MY); COUNCIL OF SCIENTIFIC AND INDUSTRIAL RESEARCH OF INDIA, Jammu Tawi (IN)

(21) Appl. No.: 12/743,179

(22) PCT Filed: Nov. 15, 2007
(86) PCT No.: PCT/MY07/00078

§ 371 Date: Jul. 22, 2010

(51) Int. Cl. A61K 36/28; A61P 37/04

(52) U.S. Cl. 424/725

(57) ABSTRACT

The present invention relates to an immunopotentiating composition and to a composition accelerating the production of interferon-[gamma]. More particularly to a process for preparation of water-soluble Labisia pumila extract and the use of said extract in a pharmaceutical preparation.

### FIELD OF INVENTION

The present invention relates to an immunopotentiating composition and to a composition accelerating the production of interferon-[gamma]. More particularly to a process for preparation of water-soluble Labisia pumila extract and the use of said extract in a pharmaceutical preparation.

### BACKGROUND OF THE INVENTION

Labisia pumila (family: Myrsinaceae), popularly known as “Kacip Fatimah”, has been used by many generations of Malay women to induce and facilitate childbirth as well as a post-partum medicine (Burkill, 1935). This study was undertaken to investigate the activity of the aqueous leaf extract from Labisia pumila leaves on innate and cellular immune responses including lymphocyte immunophenotyping, a flowcytometric technique to determine drug effect on specific surface markers present on the T cell surface (CD₄ and CD₈) and related cytokines.

A large variety of herbal drugs, have been under study for their immunomodulating, adaptogenic and rejuvenating properties. These herbal drugs are believed to promote positive health and maintain organic resistance against infections by re-establishing body equilibrium and conditioning the body tissues [Bhagwandas, Fundamentals of Ayurvedic Medicine, Bansal Co., Delhi, India, ix-xvi (1978)]. Hence the drugs of plant origin are gaining increasing popularity and are being investigated for correction of immunological disorders [Aboolenein, A. A., Back to medicinal plants therapy.

In the history of medicinal and aromatic plants; Proceedings of the second international congress. Alexandria, Egypt, ed. Abdullah, A., Hamdard Foundation Press, Pakistan, 40-44 (1980); Hikino, H., Recent research on oriental medicinal plants, In: Economic and Medicinal Plant Research Eds. Wagner, H., Hikino, H. and Farnsworth, N. R. (Academic Press, London): 53-85 (1985)].

Immunomodulation is a process, which alters the immune system of an organism by interfering with its functions. This interference results in either immunostimulation, an enhancement of immune reaction or immunosuppression that imply mainly to reduce resistance against infections and stress which may be because of environmental or chemotherapeutic factors [Patwardhan, B., Kulbag, D., Patki, P. S, and Nagsamp agi, B. A., Indian Drugs, 28 (2): 56-63 (1990)]. Immunostimulation and immunosuppression both are needed to be tackled depending on the type of immunological disturbance. Recently, search for better moieties with these activities is becoming the field of major interest. Research focussed on the development of immunomodulators is directed towards activities that can be expressed in terms of stimulation or inhibition of immune factors and their integrated functions [Labadie, R. P. Immunomodulatory compounds In: Bioactive natural products eds. Stevan M. Colegate, Russel, J. Molyneux, CRC Press Inc., 279-317 (1993)]. Recently the understanding of research on immuno-modulators has come up as a new field of immunopharmacology. Immunomodulation is an innovative strategy for overcoming incurable diseases involving cancer, AIDS, arthritis and allergies. An in-depth study of the immune system is supposed to provide both the theoretical and therapeutic background of many chronic disorders. Keeping these factors in view, major efforts have to be directed towards the formulation of new strategies, to modulate the immune responses, to permit effective treatment of various ailments associated with immune system and thus development of a safe and effective immunomodulator for clinical use has become a major goal for many pharmaceutical investigators world over.

Keeping in view the high reputation of Labisia pumila in Malaysian traditional system of medicine, it was tempting to speculate that the rejuvenating power of this plant might be due to its action on immune system of the organism as no mention is made about its such effect anywhere in literature.

### SUMMARY OF THE INVENTION

Accordingly, the object of the invention relates process for preparation of Labisia pumila extract by extracting dried Labisia pumila plant material with water to form a water-soluble extract and drying the extract obtained, characterized in that the extract having the capability to develop a composition for immunopotentiating activity. Further more, the present invention also relates to an extract obtained from Labisia pumila plant, wherein the extract having the capability of producing immunostimulating activity. The immunostimulating activity is shown to enhance Phagocytic response in-vitro of at least having 58.32% up regulated at 50 ug/ml dose. higher potentiation in cellular immune responses (T cells) where in the most significant effect was observed at the dose of 50 mg/kgorally where it showed 21.05%, 20.65% and 23.72% increase after 24 48 and 72 hours of challenge respectively against sensitized control. The extract also shows a significant increase in expression of Interleukin-2 (IL-2) a growth factor for the T cells (CD4+ and CD8+) in serum of the treated animals at 50 mg/kg per oral dose where it was 10.06%. The extract also shows a significant up-regulation of interferon-gamma (IFN-γ) where the maximum expression was observed at 50 mg/kg/oral dose where it was 12.87%. This cytokine is a signature cytokine of the Th1 response and its up regulation shows specific Th1 response. The present invention also relates to a method for treatment of immunopotentiating activity in a patient in need of such treatment, wherein the treatment includes using the said extract from obtained from Labisia pumila plant. Finally, the present invention also provides uses of the extract obtained from Labisia pumila plant for the preparation of a medicament or pharmaceutical preparations for immunopotentiating activity

### BRIEF DESCRIPTION OF THE DRAWINGS

FIG. 1 represents Effect of LPPM/A003 on Delayed type Hypersensitivity Response

FIG. 2 represents Effect of different doses of LPPM/A003 on CD4+ and CD8+ T-Cell population

FIG. 3 represents Effect of different doses of LPPM/A003 on IFN-gamma expression.

FIG. 4 represents Effect of different doses of LPPM/A003 on IL-2 expression.

FIG. 5 Effect of different doses of LPPM/A003 on IL-4 expression

### DETAILED DESCRIPTION OF THE INVENTION

The main objective of the present invention is to explore provisions of immunomodulating activity of aqueous extract from Labisia pumila plant (preferably leaves) especially on the T cell function studies. The present invention relates to immunopotentiating activity of Labisia pumila extract, having TH1 mode of T cell activation, obtained by a process of extracting the leaves of Labisia pumila with water using accelerated solvent system and drying by known methods into a free flowing powder. The extract is obtained by extracting the leaves of Labisia pumila with water using accelerated solvent system and drying by known methods into a free flowing powder code named hereafter as LPPM/A003.

The present invention is preferably used as a potent immunostimulant with broad spectrum of immunostimulant activity. Further to that the invention is also capable stimulating both specific and non-specific mechanisms. In addition the composition in the present invention enhances cell mediated component of the immune system showing enhancement in delayed type hypersensitivity response (DTH). Further more, the composition also increases CD4 and CD8+ T cell count and expression of Th1 cytokines IL-2 and IFN-gamma in antigen sensitized experimental animals. Inhibited IL-4 (Th2) expression when administered for a week

The composition also shows specific TH1 up regulation and could be a suitable for immunological effects by providing the basis for extending its use in immunodeficient states. Further more, the composition also could be suitably used as an immune modifier (potentiator) in combination therapy.

The invention will now be described in more detail by reference to the following Figures and Examples. The following examples are provided for illustrative purposes only and are not intended to limit the invention.

### Example 1

In one embodiment 1 Kg of Labisia pumila dried leaf powder is extracted with millipore/double distilled water/distilled water (1:8) and heated at 80° C. for 3 hours, the contents drained off and the solid plant material recharged with equal volume of fresh solvent (water). The process repeated three times more. All the combined extracts then concentrated on rotavapor under reduced pressure at 50° C. to give free flowing solid (extractive value ˜10%).

### Example 2

In another embodiment 1 Kg of Labisia pumila dried leaf powder is extracted with millipore/double distilled water/distilled water (1:8) and heated at 80° C. for 3 hours, the contents drained off and the solid plant material recharged with equal volume of fresh solvent (water). The process is repeated for at least three times. All the combined extracts then lyophilized to give free flowing solid (extractive value ˜9.5%).

### Example 3

In yet another embodiment, the aqueous extract of Labisia pumila prepared by taking three sets each comprising of powdered leaves (fresh or dried) with millipore/double distilled water/distilled water (1:8) using accelerated solvent extraction with temperature 40° C. for 15 minutes, the contents drained off and the solid plant material recharged with equal volume of fresh solvent (water). The process repeated three times more. All the combined extracts then concentrated on rotavapor to give free flowing solid (extractive value ˜8%).

### Example 4

In yet another embodiment, the aqueous extract of Labisia pumila prepared by taking three sets each comprising of powdered leaves (fresh or dried) with millipore/double distilled water/distilled water (1:8) using accelerated solvent extraction with temperature 60° C. for 15 minutes, the contents drained off and the solid plant material recharged with equal volume of fresh solvent (water). The process is repeated for at least three times. All the combined extracts then concentrated on rotavapor to give free flowing solid (extractive value ˜10%.

### Acute Safety Study:

The acute oral toxicity studies were carried out following OECD guidelines No. 423 in mice. The animals were observed individually after dosing at least once during the first 30 min, periodically during the first 24 h, with special attention given during the first 4 h, and daily thereafter, for a total of 14 days, simultaneously, general behaviour was also observed for 14 days. A single dose of the extract administered orally to each group of female mice did not show any change in gross general behaviour of these test animals. As this plant is already in consumption, a single dose of 5000 mg/kg p.o. was also evaluated. No mortality or any change in normal behaviour was observed at this high dose.

### Antigen (SRBC)

Fresh sheep red blood cells (SRBC) collected aseptically from the jugular vein of sheep were stored in cold sterile Alsever's solution, washed three times with pyrogen free sterile normal saline (0.9% NaCl w/v) and adjusted to a concentration of 5×10⁹ cells/ml for immunization and challenge at required time schedule.

### Phagocytic Response:

### In Vivo

The phagocytic function of the reticuloendothelial system was assayed in groups of six mice each by injecting i.v. 160 mg/kg of 1.6% suspension of gelatin stabilized carbon particles of 20-25 μm size (Atal et al., 1986). Blood samples were collected before and at intervals varying between 2 to 90 min after carbon injection. An aliquot (10 μL) of blood samples were lysed with 2 mL of 0.1% acetic acid and the transparency determined spectrophotometrically at 675 nm (Uvikon 810, spectrophotometer, Kontron Ltd, Switzerland) as per the method of Hudson and Hay (1980). LPPM/A003 showed maximum increase in carbon clearance rate from the circulation of normal animals at 50 mg/kg. An increase of 44.71% was found in Levamisole at 2.5 mg/kg orally. (Table 1).

### In Vitro

The method of Lehrer (1981) was followed. Peritoneal macrophage cells (2×10⁶) were allowed to adhere to glass cover slip for 90 minutes at 37° C. in 5% CO₂ incubator (95% humidity). Simultaneously heat killed Candida albicans cells (100° C., 60 minutes) were opsonized for 90 minutes with 20% autologous serum to promote phagocytosis. The samples were washed with PBS and immediately evaluated microscopically. The percentage and average number of Candida albicans cells (heat killed) ingested by peritoneal murine macrophages was calculated. LPPM/A003 was tested at the doses of 3.12, 6.25, 12.5 and 25 ug/ml. against phagocytic function of peritoneal macrophages. A significant increase in the phagocytosis was observed at 12.5 and 25 μg/ml. Levamisole at a concentration of 10 μg/ml showed a 65.80% increase in phagocytosis of heat killed Candida albicans by the murine macrophages (Table 1).

### Effect on Delayed Type Hypersensitivity (DTH) Response:

The method of Doherty (1981) was followed to determine SRBC induced DTH response in normal mice. Mice were immunized by injecting 20 μl of 5×10⁹ SRBC/ml subcutaneously into the right hind footpad. Animals were treated with graded doses of A003 for next six consecutive days. After seven days the thickness of the left hind foot was measured with spheromicrometer (0.01 mm pitch) and was considered as control. These mice were then challenged by injecting the same amount of SRBC intradermally into the left hind footpad. The footpad thickness was measured at 24 hr (day 1), 48 hr (day 2) and 72 hr (day 3) after challenge.

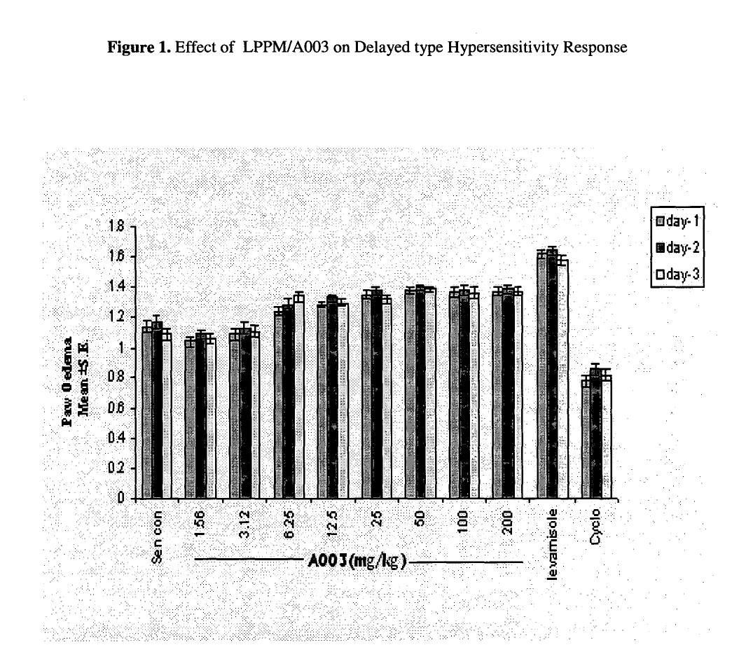

LPPM/A003 when administered orally at the oral doses of 1.56, 3.12, 6.25, 12.5, 25, 50, 100 and 200 mg/kg to normal mice showed statistically significant increase of 21.05%. in DTH response mice at a dose of 50 mg/kg in mice. The effect was also significant at 25 mg/kg p.o. dose but was less than the effect observed at 50 mg/kg p.o. dose This shows the maximum effect (CMI) to be at the dose of 50 mg/kg. (FIG. 1).

Skin allograft rejection: Mice were employed for the skin transplantation that were administered LPPM/A003 daily for 7 days and the graft rejection time (GRT) was recorded by daily observation of the epithelial survival. Oral administration of LPPM/A003 at 3.12, 6.25, 12.50, 25 50 and 100 mg/kg hastened the skin allograft rejection time. Maximum and statistically significant effect (decrease in rejection time) was observed at 50 and 100 mg/kg p.o. dose, where it was 24.38 and 26.92% respectively (Table 2). Cyclosporine at 5 mg/kg increased the rejection time by 39.69%.

### Lymphocyte Proliferation Assay to Access Mitogenic Potential

The method of Vogel G. (1996) was followed. Animals were sacrificed, their spleens removed in sterile conditions and a single cell suspension was prepared in incomplete RPMI. The viability of cells was checked. 1×10⁶ cells/ml suspension was prepared and was seeded in each well of flat bottom microtitre 96 well plates. Different concentration of LPPM/A003 was added to each well of flat bottom microtitre 96 well plate.

An aliquot of 50 μl of standard mitogens (Con-A 4 μg/ml and LPS 10 μg/ml) were added simultaneously as positive controls. The plates were incubated for 48 hours in CO₂ incubator. Absorbance was read in a multiwell plate reader at test and reference wavelengths of 540 and 620 nm respectively. The mean of the optical density of plates and percentage of each value verses control was calculated. LPPM/A003 did show proliferation of the lymphocytes but the value was within the normal range (Table 3)

### Flowcytometric Studies:

### Immunophenotyping

Phenotyping can be performed using monoclonal antibodies to the specific markers on the surface of the cells. These antibodies were first bound to a fluorescent dye, which may be fluorescence green, yellow or red, and then reacted with the lymphocytes to bind with the surface marker. The use of two different monoclonal antibodies with different fluorescing agents attached to them allows the simultaneous measurements of two clusters of differentiation (CDs) on the same cells, e.g. CD4+ and CD8+ T cells. This allows many more fluorescent cells to be counted and thus increases the accuracy of the tests.

### CD4+ and CD8+ T Cells Estimation

Mice were immunized by injecting 20 μl of 5×10⁹ SRBC/ml intraperitoneally (i.p.). Test material was administered for 7 days including the day of immunization. On day 8, the animals were challenged by injecting the same amount of SRBC i.p. in these animals, after 48 h of challenge, the blood was collected in heparinized tubes from the retroorbital plexuses. In this study FITC labelled anti-mouse CD4+ monoclonal antibody and phycoerythrin (PE) labelled CD8+ monoclonal antibody was used. CD4+ antibody reacts with CD4+ differentiation antigen expressed on MHC class II restricted T cells that includes most helper cells (CD4+), whereas PE labelled CD8+ antibody reacts with CD8+ differentiation antigen present on MHC class I restricted T cells. These were used to determine the percentage of CD4+ and CD8+ T cells in the control and treated groups of animals.

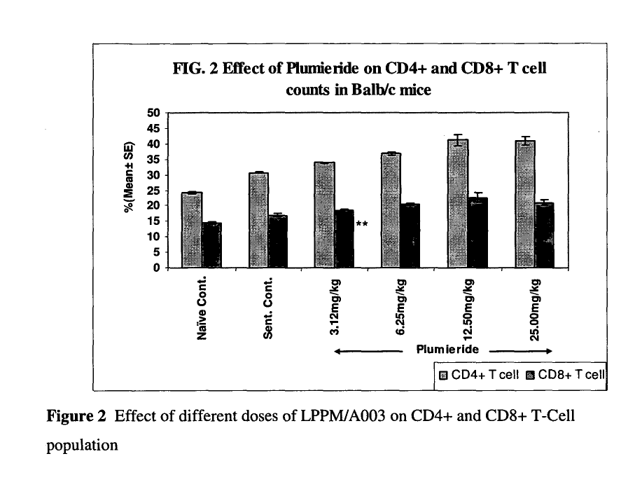

The animals treated with LPPM/A003 at 1.56, 3.12, 6.25, 12.5, 25, 50, 100 and 200 mg/kg p.o. dose showed a maximum effect of 31.98% (percent mean) of CD4⁺ and 21.84% of CD8⁺ T cells at 50 mg/kg p.o. dose. The effect at 25 mg/kg oral dose was 31.7% of CD4⁺ and 21.39% of CD8⁺ T cells. The control values were 23.12% of CD4+ and 14.02% of CD8⁺ T cells. This shows an increase in CD4+ and CD8⁺ T cell count (FIG. 2). Test material exhibited increase in CD4+ and CD8+ counts in normal mice. CD4 and CD8 are the T cell surface markers for Th1/Th2 and Tc subsets of T lymphocytes respectively. CD4+ molecules are considered central to regulation of classical cell mediated functions such as delayed-type hypersensitivity (DTH) response and B cell activation.

### Estimation of Intracellular Cytokines

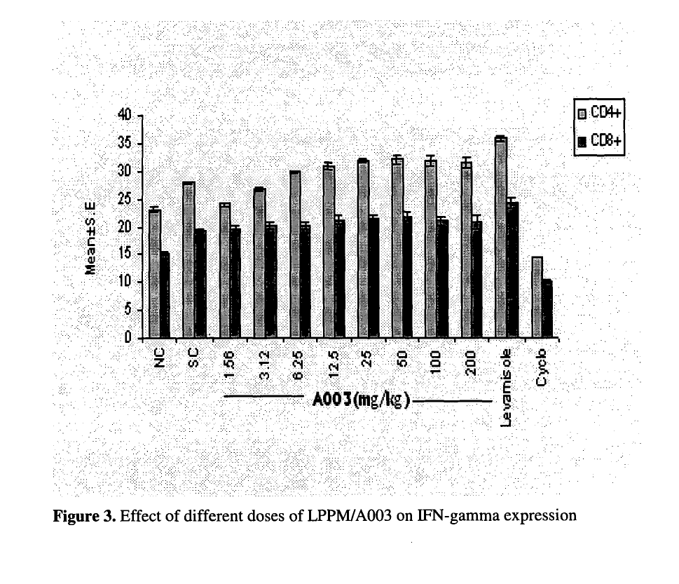

The blood was collected in heparinised tubes from retroorbital plexus of the animal. FITC-labeled anti-mouse CD4+ monoclonal antibody and phycoerythrin (PE)-labeled IL-2, IFN-γ and IL-4 monoclonal antibodies were used in experimentation. The percentage of intracellular IFN-γ in CD4+ T cell is 11.92±1.00 and 11.86±1.00 at both 50 and 25 mg/kg p.o dose. The sensitized control group showed 10.56±0.32 of intracellular IFN-γ and normal non-sensitized group values were 7.11±0.16 of IFN-γ in CD4+ T cells. This shows increase in the treated groups with maximum effect at 50 mg/kg p.o. dose (FIG. 3).

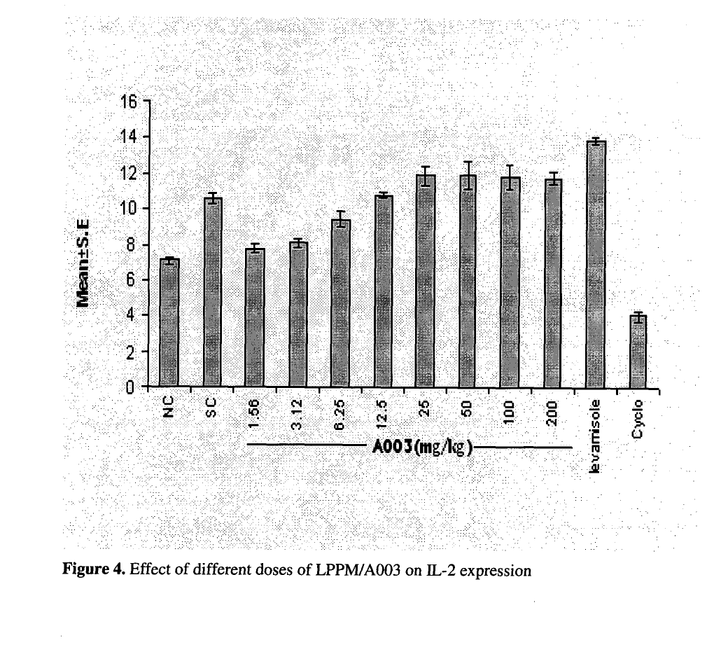

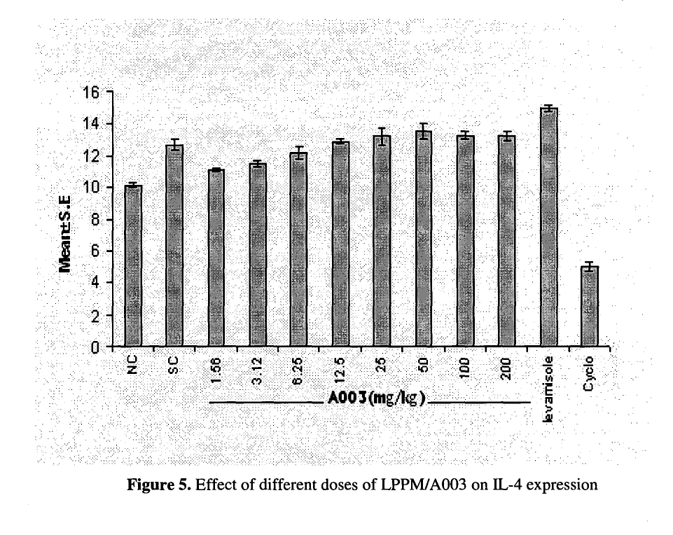

The maximum increased expression of IL-2 by A003 was observed at 50 mg/kg p.o. dose level where it was found to be 13.49±0.2 (% mean±S.E.) in CD4⁺ T cells. The percentage of IL-2 in the control (sensitized) group and in non-sensitized control group was 12.62±0.11 and 10.10±0.13 respectively (FIG. 4). LPPM/A003 induced a dose related increased expression of IL-2 (interleukin-2) production by CD4+ T helper cells. IL-2 promotes proliferation and differentiation of additional CD4+ T cells, B cells, and activates macrophages. Due to its effects on T cells and B cells, IL-2 is a central regulator of immune responses. It also possibly induces the stimulation of the synthesis of interferon gamma (IFN-γ) by the T cells (TH1 response) LPPM/A003 showed inhibition of IL-4 expression with maximum decreased expression at 50-mg/kg p.o. dose level where it was found to be 5.27±0.44 (% mean±S.E.) in CD4⁺ T cells. The percentage of IL-4 in the control (sensitized) group and in non-sensitized control group was 8.62±0.13 and 6.24±0.32 respectively (FIG. 5).

TABLE 1
Effect of LPPM/A003 on Phagocytic function

| | % Phagocytosis | | % Phagocytosis |
|---|---|---|---|
| Treatment | In vitro study | Treatment | Ex -In vivo study |
| dose μg/ml | Mean ± S.E. | dose mg/ml p.o. | Mean ± S.E. |
| control | 26.00 ± 2.32 | control | 1.23 ± 0.14 |
| 12.5 | 28.50 ± 1.95 | 12.5 | 1.26 ± 0.12 |
| | (9.61↑) | | (2.43↑) |
| 25 | 30.12 ± 2.11 | 25 | 1.45 ± 0.12 |
| | (15.84↑) | | (17.88↑) |
| 50 | 41.16 ± 0.40*** | 50 | 1.63 ± 0.16* |
| | (58.30↑) | | (32.52↑) |
| 100 | 40.23 ± 3.24*** | 100 | 1.69 ± 0.12** |
| | (54.73↑) | | (37.39↑) |
| 200 | 37.22 ± 3.39** | 200 | 1.59 ± 0.08* |
| | (43.15↑) | | (29.26↑) |
| Levamisole 10 | 43.11 ± 2.92*** | 2.5 | 1.78 ± 0.11*** |
| | (65.80↑) | | (44.71↑) |

n = 6
Data are represented as mean ± S.E;
*p < 0.05,
**p < 0.01,
***p < 0.001
Figures in parenthesis represents percentage change

TABLE 2
Effect of LPPM/A003 on skin allograft
rejection (CMI reponse) in mice

| | | Rejection Time | |
|---|---|---|---|
| | Dose mg/kg | Days | Graft Rejection |
| Treatment | p.o. | Mean ± S.E. | (Percent) |
| Control | — | 13.00 ± 0.25 | — |
| LPPM/A003 | 25.00 | 11.12 ± 0.11 | 14.46 ↑ |
| LPPM/A003 | 50.00 | 9.83 ± 0.16 | 24.38^{a} ↑ |
| LPPM/A003 | 100.00 | 9.50 ± 0.22 | 26.92^{a} ↑ |
| Levamisole | 2.5 | 8.50 ± 0.22 | 34.61^{b} ↑ |
| Cyclosporin | 5.00 | 18.16 ± 0.16 | 39.69^{b} ↓ |

n = 6
Data is represented as Mean ± S.E.
↑: Hastened; ↓: Delayed.
^{a}P < 0.01,
^{b}P < 0.001;
Student's ‘t’ test

TABLE 3
Mitogenic effect of Labisia on murine spleenocytes by
lymphocyte proliferation assay

| | | | | | % |
|---|---|---|---|---|---|
| | | | Conc. | O.D. at (460 nm) | Mitogenic |
| S. No. | Sample | | μg/ml | Mean ± S.E. | response |
| | | LPS | | | |
| | | 10 μg/ml | | | |
| 1 | Medium | — | — | 0.124 ± 0.002 | — |
| 2 | Cells + Medium | — | — | 0.279 ± 0.010 | — |
| 3 | LPPM/A003 | — | 100 | 0.938 ± 0.041 | 236.20↑ |
| 4 | LPPM/A003 | — | 30 | 0.752 ± 0.028 | 169.53↑ |
| 5 | LPPM/A003 | — | 10 | 0.629 ± 0.018 | 125.44↑ |
| 6 | LPS control | 50 μl | — | 1.047 ± 0.093 | 275.26↑ |
| 7 | LPS + LPPM/A003 | 50 μl | LPS + 100 | 0.959 ± 0.077 | 243.72↑ |
| 8 | LPS + LPPM/A003 | 50 μl | LPS + 30 | 0.784 ± 0.025 | 181.00↑ |
| 9 | LPS + LPPM/A003 | 50 μl | LPS + 10 | 0.733 ± 0.037 | 162.72↑ |
| | | CON-A | | | |
| | | 10 μg/ml | | | |
| 1 | Medium | — | — | 0.124 ± 0.002 | — |
| 2 | Cells + Medium | — | — | 0.279 ± 0.010 | — |
| 3 | LPPM/A003 | — | 100 | 0.844 ± 0.044 | 202.50↑ |
| 4 | LPPM/A003 | — | 30 | 0.790 ± 0.019 | 183.15↑ |
| 5 | LPPM/A003 | — | 10 | 0.774 ± 0.019 | 177.41↑ |
| 6 | ConA control | 50 μl | — | 0.896 ± 0.030 | 221.14↑ |
| 7 | ConA + LPPM/A003 | 50 μl | ConA + 100 | 0.883 ± 0.032 | 216.14↑ |
| 8 | ConA + LPPM/A003 | 50 μl | ConA + 30 | 0.855 ± 0.011 | 206.45↑ |
| 9 | ConA + LPPM/A003 | 50 μl | ConA + 10 | 0.841 ± 0.023 | 201.14↑ |

Labisia pumila aqueous extract (LPPM/A003) on submitting to immunopharmacological screening showed significant immunopotentiating activity. It produced a dose related increase in the clearance of carbon particles from the reticuloendothelial system and also the hastening in the rate of phagocytosis in vitro by marine macrophages, thereby suggesting an increase in the functioning of macrophages (innate response) by causing stimulation of non-specific immune response (Table 1).

In T cell specific response reaction (cell mediated immunity) it showed the stimulatory effect on ‘T’ lymphocytes in SRBC induced DTH response (FIG. 1). An evidence, that leads to support hypothesis of T-lymphocytes stimulation by is the reduction in the homologous skin graft rejection time in mice treated with LPPM/A003 (Table 2). The basic mechanism involved in reduction in graft rejection time is the stimulation of T-lymphocytes i.e. CD₄ and CD₈ positive T cells (FIG. 2). One of the major effector functions of CD4+ T cells is the activation of macrophages and this plays an important role in enhancing the activity at sites of insult. CD4+ T cell activation by LPPM/A003 may be one of the factors responsible for the increase in the functioning of the macrophages.

T helper (Th) lymphocyte homeostasis is crucial in orchestrating the appropriate cytokine responses and hence remains as one of targets for immunomodulation. T helper cells have two subsets known as Th1 and Th2, and the cytokines they produce are known as Th1-type cytokines and Th2-type cytokines. Th1-type, cytokines (IFN-gamma, IL-2) promotes cell-mediated immunity responsible for killing intracellular parasites while Th2-type cytokines (interleukins 4) are associated with humoral immunity. These subsets involve fundamentally different and opposing effector functions and extreme of either leads to disease. Hence, the optimal immunotherapy should restore or maintain a well balanced Th1 and Th2 response, suited to the immune challenge (Mosmann and Coffman, 1989). A variety of agents that selectively boost either Th1 or Th2 responses have been studied including small molecular weight synthetic compounds, oligodeoxynucleotides, extracts from fungi or bacterium metal composite and Japanese-Chinese herbal medicine.

The trends indicate that agents, which selectively modulate either Th1 or Th2 responses, may provide means of achieving T cell homeostasis. (Patwardhan and Gautam, 2005). Th1/Th2 balance concept considers that immune response is usually polarized to give predominantly either a Th1 or a Th2 response and fine-tuning of inhibitors, activators and regulatory signals ensures immunostasis. Currently, much of the literature supports Th1/Th2 balance concept to the level of paradigm and many of T cell directed therapies have provided modest clinical benefits. Th1 response primarily promotes cytolytic T cells (CTLs), which are important in responding to infections. Cytokines play a central role in the regulation of hematopoises, mediating the differentiating migration, activation and proliferation of phenotypically diverse cells (Mossmann & Fong, 1989; Constant & Bottomly, 1997). Many cytokines are pleiotropic and possess overlapping functions thus regulating the production of other cytokines and make-up of the cytokine milieu (in vivo) is often of a greater importance than the actions of a single cytokine. The analysis and quantification of cytokines in biological fluids has become a widely used procedure in research and clinical laboratories and is clearly important in furthering our understanding of many immunological functions. The results suggest that oral administration of LPPM/A003 induced a dose related increased expression of interferon gamma (IFN-γ) and IL-2 (interleukin-2) (FIGS. 3 and 4). It, however, suppressed the expression of IL-4 (FIG. 5) that may possibly be due to the cross effects of CD4+ T cells secreted cytokines, thus expressing specific Th1 response. LPPM/A003 did not have any cyto-toxicity and both T and B cell proliferation was within normal range.

In conclusion, this study suggests that even complex botanical mixtures can exhibit selectivity in immune therapy and will be useful to underline importance of systems approaches in the ethnopharmacology based drug discovery (Verpoorte et al., 2005; Patwardhan, 2000). Such immunoactive mixtures may deliver appropriate synergistic moieties, which concurrently or simultaneously modulate immune matrix and restores homeostatic conditions. This study establishes Th1 up-regulating activity of LPPM/A003 using flowcytometry and suggests its use in conditions where Th1/Th2 modulation is required Such agents are being sought for the treatment of infectious diseases, immunodeficient diseases, or for generalized immunosuppression induced by drug treatment; for combination therapy with antibiotics; and as adjuncts for vaccines. All these findings suggest that LPPM/A003 has a significant immunostimulant activity suggestive of its possible usefulness as a therapeutic agent in immune compromised patients.

1. A process for preparation of Labisia pumila extract by extracting dried Labisia pumila plant material with water to form a water-soluble extract and drying the extract obtained, wherein the extract has the capability to develop a composition for immunostimulating activity.

2. An extract obtained from Labisia pumila plant produced according to claim 1, the extract having the capability of producing immunostimulating activity.

3. The extract as claimed in claim 2, wherein the extract having a higher potentiation in cellular immune responses (T cells) wherein the most significant effect was observed at the dose of 50 mg/kg orally, where it showed 21.05%, 20.65% and 23.72% increase after 24, 48 and 72 hours of challenge respectively against sensitized control.

4. The extract as claimed in claim 2, wherein the extract has a significant increase in expression of Interleukin-2 (IL-2) a growth factor for the T cells (CD4+ and CD8+) in serum of the treated animals at 50 mg/kg per oral dose where it was 10.06%.

5. The extract as claimed in claim 2 wherein the extract has a significant up-regulation of interferon-gamma (IFN-γ) wherein the maximum expression was observed at 50 mg/kg/oral dose where it was 12.87%, wherein cytokine is a signature cytokine to Th1 response and has a regulation showing substantially specific Th1 response.

6. The use of the extract obtained from Labisia pumila plant according to claim 2 for the preparation of a medicament or pharmaceutical preparation capable of producing immunostimulating activity.

7. The extract as claimed in claim 2, wherein the immunostimulating activity is shown to enhance Phagocytic response in vitro of at least having 58.32% up regulated at 50 μg/ml dose.

8. The use of the extract obtained from Labisia pumila plant according to claim 7 for the preparation of a medicament or pharmaceutical preparation capable of producing immunostimulating activity.
